(19) United States
(12) Patent Application Publication
(10) Pub. No.: US 2022/0087577 A1
(43) Pub. Date: Mar. 24, 2022

(54) METHOD, APPARATUS AND PROGRAM FOR MEASURING ELECTROCARDIOGRAM-BASED BLOOD GLUCOSE USING ARTIFICIAL INTELLIGENCE

(71) Applicant: Iplemind.inc, Seoul (KR)

(72) Inventors: Seoung Han Lee, Seoul (KR); Seung Hee Baik, Bucheon-si (KR)

(73) Assignee: Iplemind.inc, Seoul (KR)

(21) Appl. No.: 17/478,206

(22) Filed: Sep. 17, 2021

(30) Foreign Application Priority Data
Sep. 18, 2020 (KR) 10-2020-0120372
Nov. 6, 2020 (KR) 10-2020-0147609

(51) Int. Cl. A61B 5/145; A61B 5/00; A61B 5/318

(52) U.S. Cl. CPC A61B 5/14532; A61B 5/318; A61B 5/7285; A61B 5/7264

(57) ABSTRACT

A method for measuring an electrocardiogram-based blood glucose using artificial intelligence is provided. The method includes: receiving an electrocardiogram signal of a user; extracting a plurality of unit electrocardiogram signals from the received electrocardiogram signal; extracting a blood glucose spatial feature from each of the plurality of unit electrocardiogram signals using a first artificial neural network model; and extracting a blood glucose feature by analyzing a time series change of the blood glucose spatial feature using a second artificial neural network model, and predicting blood glucose of the user based on the extracted blood glucose feature.

### CROSS-REFERENCE TO RELATED APPLICATION(S)

This application claims benefit of priority to Korean Patent Application No. 10-2020-0120372 filed on Sep. 18, 2020 and Korean Patent Application No. 10-2020-0147609 filed on Nov. 6, 2020 in the Korean Intellectual Property Office, the disclosure of which is incorporated herein by reference in its entirety.

### BACKGROUND

### 1. Field

The present invention relates to a method, apparatus, and program for measuring electrocardiogram-based blood glucose using artificial intelligence, and more particularly, to a method, apparatus, and program for measuring blood glucose based on artificial intelligence that predicts blood glucose of a user based on an electrocardiogram (ECG) measured from the user.

### 2. Description of Related Art

The number of diabetic patients is rapidly increasing in the era of changes in diet and aging, and medical expenses are also increasing accordingly.

Diabetes is a disease caused by high levels of glucose (blood glucose) in the blood, and refers to a disease state in which insulin hormone is required for glucose absorbed into the blood to be used by cells, but glucose may not be used by cells due to insufficient insulin and insulin resistance of the pancreas, and is accumulated in the blood and discharged through the urine.

The diabetes is a very dangerous disease that may lead to acute or chronic complications and, in severe cases, death, when blood glucose is not properly managed.

The acute complication is a condition in which the blood glucose rapidly rises or falls too much due to poor blood glucose control, and when hyperglycemia persists, dehydration occurs due to an osmotic diuretic effect, when the loss of body fluid is accelerated, it may lead to loss of consciousness, and in the case of hypoglycemia, it may lead to confusion of consciousness, behavioral disturbance, and, in severe cases, death.

The chronic complication is mainly caused by broken blood vessels and includes various diseases such as diabetic retinopathy of the eye, damage to peripheral nerves and autonomic nerves, arteriosclerosis of blood vessels in the legs, and diabetic foot lesions.

Until now, since there is no treatment method to return the pancreas to normal after the diabetes occurs, managing the blood glucose within a normal range through continuous measurement of the blood glucose is a realistic alternative.

In accordance with the rapid increase in the number of patients with diabetes, which is a representative example of a chronic disease, many personal blood glucose meters have been developed and sold on the market.

Among these, the conventional invasive blood glucose measurement method is a method in which blood is collected by piercing a skin with a needle and measured with a blood glucose meter.

However, the invasive blood glucose measurement method has a disadvantage of giving pain and discomfort to the patient as the patient has to collect blood several times a day, and as a result, there is a problem in that the patients with diabetes may be exposed to the risk of complications because patients with diabetes may not be expected to constantly measure their blood glucose.

### SUMMARY

An object of the present invention is to provide a method, apparatus, and program for measuring blood glucose based on artificial intelligence that predicts blood glucose of a user from an electrocardiogram (ECG) of the user using a learned artificial neural network model.

According to an exemplary embodiment of the present invention, a method for measuring electrocardiogram-based blood glucose using artificial intelligence includes: receiving an electrocardiogram signal of a user; extracting a plurality of unit electrocardiogram signals from the received electrocardiogram signal; extracting a blood glucose spatial feature from each of the plurality of unit electrocardiogram signals using a first artificial neural network model; and extracting a blood glucose feature by analyzing a time series change of the blood glucose spatial feature using a second artificial neural network model, and predicting blood glucose of the user based on the extracted blood glucose feature.

The first artificial neural network model may be a convolution neural network (CNN) model, and the second artificial neural network model may be a recurrent neural network (RNN) model.

The convolution neural network model may extract the blood glucose spatial feature, which is a spatial feature related to a blood glucose level and/or a blood glucose change, from a waveform of each of the plurality of unit electrocardiogram signals.

The number of convolution neural networks of the convolution neural network model may coincide with the number of the plurality of extracted unit electrocardiogram signals.

The recurrent neural network model may extract a blood glucose feature vector representing a time-series change of a blood glucose spatial feature of each of a plurality of unit electrocardiogram images extracted through the plurality of convolution neural networks, and synthesize the extracted blood glucose features to extract the blood glucose feature.

The number of recurrent neural networks of the recurrent neural network model may coincide with the number of convolution neural networks.

The predicted blood glucose may be a blood glucose level value and/or a blood glucose state value.

According to another exemplary embodiment of the present invention, an apparatus for measuring electrocardiogram-based blood glucose using artificial intelligence includes: an electrocardiogram signal receiving unit configured to receive an electrocardiogram signal of a user and extract a plurality of unit electrocardiogram signals from the received electrocardiogram signal; and an AI processor configured to extract a blood glucose spatial feature from each of the plurality of unit electrocardiogram signals using a first artificial neural network model, extract a blood glucose feature by analyzing a time series change of the blood glucose spatial feature using a second artificial neural network model, and predict blood glucose of the user based on the extracted blood glucose feature.

According to still another exemplary embodiment of the present invention, a user-customized healthcare service system includes: an electrocardiogram measuring device configured to measure an electrocardiogram of a user; an electrocardiogram-based blood glucose apparatus configured to receive an electrocardiogram signal of the user from the electrocardiogram measuring device, extract a plurality of unit electrocardiogram signals from the received electrocardiogram signal, and predict blood glucose of the user from the plurality of unit electrocardiogram signals using a trained artificial neural network model; and a server configured to provide an emergency dispatch service to a location of the user or provide a user-customized prescription service when the blood glucose of the user measured by the electrocardiogram-based blood glucose measuring apparatus is in a dangerous state, wherein the electrocardiogram-based blood glucose measuring apparatus is configured to extract a blood glucose spatial feature from each of the plurality of unit electrocardiogram signals using a first artificial neural network model, extract a blood glucose feature by analyzing a time series change of the blood glucose spatial feature using a second artificial neural network model, and predict blood glucose of the user based on the extracted blood glucose feature.

According to still another exemplary embodiment of the present invention, a program stored in a recording medium in which a program code for executing the method for measuring electrocardiogram-based blood glucose described above is recorded may be provided.

According to the present invention, by presenting the artificial neural network model trained to extract the features from the electrocardiogram signals of the user and predict the blood glucose of the user based on the extracted features, the blood glucose of the user may be more easily and quickly predicted.

In addition, the present invention may be applied to a health care system including an electrocardiogram measuring device, thereby providing a customized health care service to a user. In other words, the diabetes is a disease that requires lifelong management after onset, and according to the present invention, when the user is wearing the electrocardiogram measuring device, the healthcare system may perform a function of providing the alarm to the user or automatically reporting to an emergency reporting agency in case of an emergency by predicting the blood glucose of the user based on the electrocardiogram of the user measured in real time.

### BRIEF DESCRIPTION OF DRAWINGS

The above and other aspects, features and other advantages of the present disclosure will be more clearly understood from the following detailed description taken in conjunction with the accompanying drawings, in which:

FIGS. 1(a) and 1(b) are diagrams illustrating an electrocardiogram measuring device according to an exemplary embodiment of the present invention.

FIG. 2 is a diagram illustrating an electrocardiogram signal according to an exemplary embodiment of the present invention.

FIG. 3 is a flowchart illustrating a method of training an artificial neural network model using training data according to an exemplary embodiment of the present invention.

FIG. 4 is a flowchart illustrating an electrocardiogram-based blood glucose measuring method using artificial intelligence according to an exemplary embodiment of the present invention.

FIG. 5 is a diagram illustrating an artificial neural network model according to an exemplary embodiment of the present invention.

FIG. 6 is a diagram illustrating a relationship between an electrocardiogram signal and blood glucose according to an exemplary embodiment of the present invention.

FIG. 7 is a block diagram illustrating an electrocardiogram-based blood glucose measuring apparatus using artificial intelligence according to an exemplary embodiment of the present invention.

FIG. 8 is a block diagram illustrating a customized healthcare service system according to an exemplary embodiment of the present invention.

### DETAILED DESCRIPTION

The following description merely illustrates the principles of the present invention. Therefore, those skilled in the art may implement the principle of the present invention and invent various devices included in the spirit and scope of the present invention, although not clearly described or illustrated in the present specification. In addition, it is to be understood that all conditional terms and exemplary embodiments mentioned in the present specification are obviously intended only to allow those skilled in the art to understand a concept of the present invention in principle, and the present invention is not limited to exemplary embodiments and states particularly mentioned as such.

Further, it is to be understood that all detailed descriptions mentioning specific exemplary embodiments of the present invention as well as principles, aspects, and exemplary embodiments of the present invention are intended to include structural and functional equivalences thereof. Further, it is to be understood that these equivalences include an equivalence that will be developed in the future as well as an equivalence that is currently well-known, that is, all elements invented so as to perform the same function regardless of a structure.

Therefore, it is to be understood that, for example, a block diagram of the present specification shows a conceptual aspect of an illustrative circuit for embodying the principle of the present invention. Similarly, it is to be understood that all flowcharts, state transition diagrams, pseudo-codes, and the like, illustrate various processes that maybe tangibly embodied in a computer readable medium and that are executed by computers or processors regardless of whether or not the computers or the processors are clearly illustrated.

Functions of various elements including processors or functional blocks represented as concepts similar to the processors and illustrated in the accompanying drawings may be provided using hardware having the capability to execute appropriate software as well as dedicated hardware. When the functions are provided by the processors, they may be provided by a single dedicated processor, a single shared processor, or a plurality of individual processors and some thereof may be shared with each other.

In addition, terms mentioned as a processor, a control, or a concept similar to the processor or the control should not be interpreted to exclusively cite hardware having the capability to execute software, but should be interpreted to implicitly include digital signal processor (DSP) hardware and a read only memory (ROM), a random access memory (RAM), and a non-volatile memory for storing software without being limited thereto. The above-mentioned terms may also include well-known other hardware.

In the claims of the present specification, components represented as means for performing functions mentioned in a detailed description are intended to include all methods for performing functions including all types of software including, for example, a combination of circuit elements performing these functions, firmware/micro codes, or the like, and are coupled to appropriate circuits for executing the software so as to execute these functions. It is to be understood that since functions provided by variously mentioned means are combined with each other and are combined with a scheme demanded by the claims in the inventions defined by the claims, any means capable of providing these functions are equivalent to means recognized from the present specification.

The above-mentioned objects, features, and advantages will become more obvious from the following detailed description provided in relation to the accompanying drawings. Therefore, those skilled in the art to which the present invention pertains may easily practice a technical idea of the present invention. Further, in describing the present invention, in the case in which it is judged that a detailed description of a well-known technology associated with the present invention may unnecessarily make the gist of the present invention unclear, it will be omitted.

Hereinafter, various exemplary embodiments of the present invention will be described in detail with reference to the accompanying drawings.

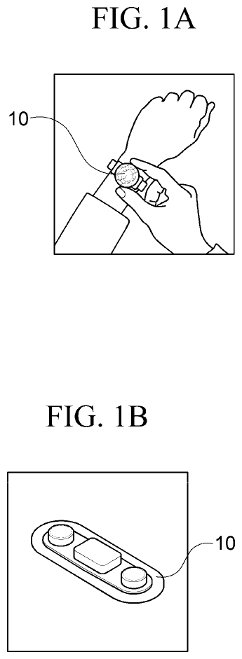

FIGS. 1(a) and 1(b) are diagrams illustrating an electrocardiogram measuring device according to an exemplary embodiment of the present invention. Referring to FIGS. 1(a) and 1(b), an electrocardiogram measuring device 10 may be implemented as a wearable device that may be worn on or attached to a body of a user, and may be implemented as, for example, a watch type that maybe worn on a wrist of the user as illustrated in FIG. 1(a) or a patch type that may be attached to the body of the user as illustrated in FIG. 1(b).

However, the electrocardiogram measuring device 10 is not limited thereto, and may have various shapes such as a bracelet, a band, a ring, a necklace, an anklet, or a waistband.

The electrocardiogram measuring device 10 may measure an electrocardiogram, which is an electric potential related to a heartbeat appearing on a body surface when electrodes provided in the device 10 come into contact with a user's skin.

As an example, in the case in which the electrocardiogram measuring device 10 is implemented as illustrated in FIG. 1(a), when the user wears the wearable device on one hand and touches one electrode of the wearable device with the other hand, an electrical closed circuit is formed inside the body of the user, so that a change in current due to a heartbeat may be measured as a change in an electrocardiogram.

The electrocardiogram measured by the electrocardiogram measuring device 10 includes information on electrical activity of the heart.

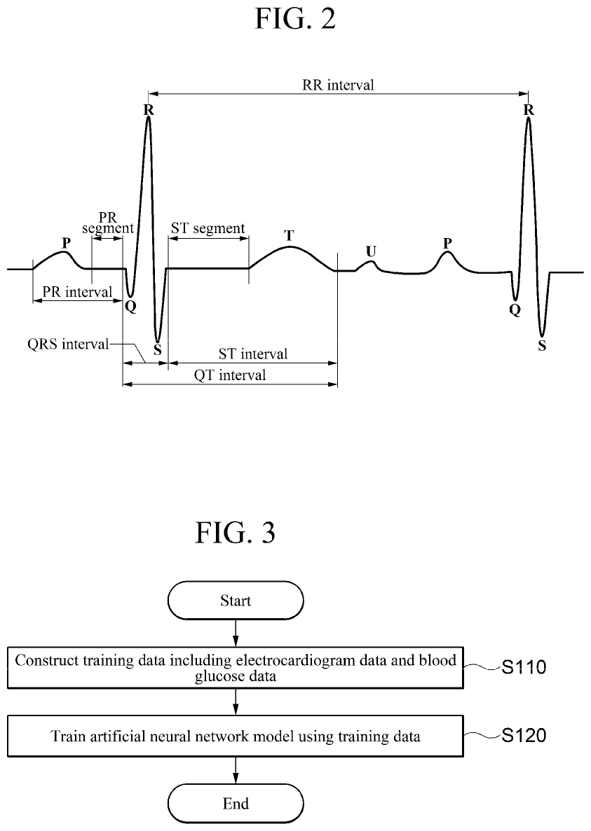

FIG. 2 is a diagram illustrating an electrocardiogram signal according to an exemplary embodiment of the present invention. The electrical activation phase of the heart is largely divided into atrial depolarization, ventricular depolarization, and ventricular repolarization phases, and these phases are reflected in the form of waves called P, QRS, and T waves.

That is, the electrocardiogram signal measured by the electrocardiogram measuring device 10 may include the P wave, the QRS wave, and the T wave, and an X axis means time and a Y axis means voltage.

It is possible to extract unique features for each person by analyzing the continuous P wave, Q wave, R wave, S wave, and T wave of the electrocardiogram signal and the QRS waveform (QRS Complex), which is a waveform that changes the most.

The PQRST waveform of the electrocardiogram signal includes various pieces of information about the electrical activity of the heart.

As an example, the PR interval represents a time interval between the P wave and the start of the QRS complex, a PR segment represents a time interval from an end of the P wave to the start of the QRS complex, a QT interval represents a time interval between the start of the Q wave and an end of the T wave, an ST segment represents a time interval between an end of the S wave and the start of the T wave, an ST interval represents the time interval between the end of the S wave and the end of the T wave, a QRS interval represents the time interval between the start of the QRS complex and an end of the QRS complex, and an RR interval represents the time interval between a peak of the R wave and a peak of a next R wave.

As described above, the electrocardiogram signal measured by the electrocardiogram measuring device 10 includes information on the electrical activity of the heart, and the measured electrocardiogram signal may be used to predict blood glucose according to the method according to the present invention.

More specifically, according to the present invention, the blood glucose of the user may be predicted by analyzing the electrocardiogram signal measured by the electrocardiogram measuring device 10 worn on or attached to the user using the trained artificial neural network model. This will be described later with reference to the drawings.

FIG. 3 is a flowchart illustrating a method of training an artificial neural network model using training data according to an exemplary embodiment of the present invention. Referring to FIG. 3, first, in order to train the artificial neural network model, training data including electrocardiogram data and blood glucose data may be constructed (S110).

Here, the electrocardiogram data may be an electrocardiogram signal image displayed as a voltage value according to the time as illustrated in FIG. 2. In addition, the blood glucose data may include a blood glucose level value and/or “a blood glucose state value including of high blood glucose, low blood glucose, medium blood glucose, and normal blood glucose”.

For example, the high blood glucose may refer to a state in which a fasting blood glucose level is 126 mg/dl or more and a blood glucose level 2 hours after a meal is 200 mg/dl or more.

In addition, the low blood glucose may refer to a state in which the blood glucose level falls below 70 mg/dl. In addition, the medium blood glucose is a state in which the fasting blood glucose level has a value between 100 and 126 mg/dl, and may refer to a state in which there is a possibility of developing the high blood glucose. In addition, the normal blood glucose may refer to a state in which the fasting blood glucose level is generally less than 70 to 100 mg/dl.

Here, the training data may include electrocardiogram data and blood glucose data at the same time point.

Specifically, the electrocardiogram signal may include a plurality of unit electrocardiogram signals according to the heartbeat of the user. That is, the electrical activation phase of the heart according to one heartbeat is divided into atrial depolarization, ventricular depolarization, and ventricular repolarization, and one unit electrocardiogram signal may be formed through the processes of atrial depolarization, ventricular depolarization, and ventricular repolarization. The unit electrocardiogram signal may include a P wave, a QRS wave, and a T wave.

In this case, the training data may include a first unit electrocardiogram signal and first blood glucose data measured in a first time interval, a second unit electrocardiogram signal and second blood glucose data measured in a second time interval, and an N-th electrocardiogram signal and N-th blood glucose data measured in an N-th time interval.

The training data is data for training the artificial neural network model, and the artificial neural network model trained using the training data may determine an association between the electrocardiogram and blood glucose.

To this end, according to the present invention, it is possible to acquire the training data by performing an electrocardiogram measurement simultaneously with continuous glucose monitors (CGM) and an oral glucose tolerance test.

In this case, the electrocardiogram data of the user and the blood glucose data related thereto may be acquired through the following processes.

[Oral Glucose Tolerance Test and Electrocardiogram Measurement]

Oral glucose tolerance test and electrocardiogram measurement are performed simultaneously to measure an electrocardiogram according to changes in blood glucose.

As a measurement method, after attaching an electrocardiogram measuring device to the body of a user and maintaining a fasting state for at least 8 hours, the user drinks 75 g of glucose dissolved in 250 to 300 ml of water over 5 minutes, and blood is then drawn at 30, 60, and 120 minutes to measure concentration of the blood glucose.

[Continuous Glucose Monitors and Electrocardiogram Measurement]

Continuous glucose monitors and electrocardiogram measurement are simultaneously performed for a living blood glucose management model, and as a measurement method, after attaching a continuous glucose measuring device and an electrocardiogram measuring device to the body of the user, the user spends 7 days in daily life, and the electrocardiogram of the user is measured according to changes in blood glucose.

Through such processes, it is possible to construct an electrocardiogram data set according to the change in blood glucose level, and the electrocardiogram data set may be used as training data for training the artificial neural network model. As an example, training data in which a blood glucose level value, a blood glucose state value, and time sequence information are labeled on each of a plurality of unit electrocardiogram signals continuously measured over time may be constructed.

In addition, the above-described training data may be constructed for each user.

Thereafter, the artificial neural network model may be trained using the acquired training data (S120). Here, the artificial neural network model is a statistical model that mimics a biological neural network, and may have a problem-solving ability through a training process.

Parameters of the artificial neural network model may be adjusted through a training process using training data.

Here, the artificial neural network model maybe trained by a deep learning method. The deep learning method refers to a machine learning algorithm that attempts high-level abstraction through a combination of several nonlinear transformation techniques.

For example, the artificial neural network model according to the present invention may be trained using a supervised learning method. In addition, the artificial neural network model according to the present invention may be trained through unsupervised learning of finding a determination criterion by performing self-training using the training data without any supervision. In addition, the artificial neural network model according to the present invention may be trained through reinforcement learning by using a feedback as to whether a result of the situation determination according to the learning is correct. In addition, the artificial neural network model according to the present invention may be trained using a training algorithm including error back-propagation or gradient descent.

Here, the artificial neural network model according to the present invention may be a combination of a convolution neural network (CNN) model and a recurrent neural network (RNN) model.

The convolution neural network (CNN) model may not be suitable for predicting patterns that change over time because it does not consider past information and perform classification using feature values included in current input data. However, the convolution neural network (CNN) model is suitable for training hierarchical and abstract representations of inputs related to the performance of a specific task.

In addition, the recurrent neural network (RNN) model is suitable for training time-series correlation because it includes a cyclic connection structure for reflecting past output values to current input data operations.

Accordingly, according to the present invention, it is possible to train the artificial neural network model in which the CNN model and the RNN model are combined so that the electrocardiogram electrode waveform identified and imaged by the sensor and the blood glucose level value identified and digitized by the sensor are matched with each other, and it is possible to minimize an error in a bio-signal by calculating a result value from values classified through the trained artificial neural network model.

In addition, according to the present invention, decision tree modeling may be performed so that an alarm is generated according to an emergency event definition when the bio-signal value is deviated from an absolute value range thereof by designating the absolute value range for each bio-signal value.

Meanwhile, the artificial neural network model trained using the training data according to the above-described process may receive the electrocardiogram of the user and predict the blood glucose of the user. This will be described later with reference to the drawings.

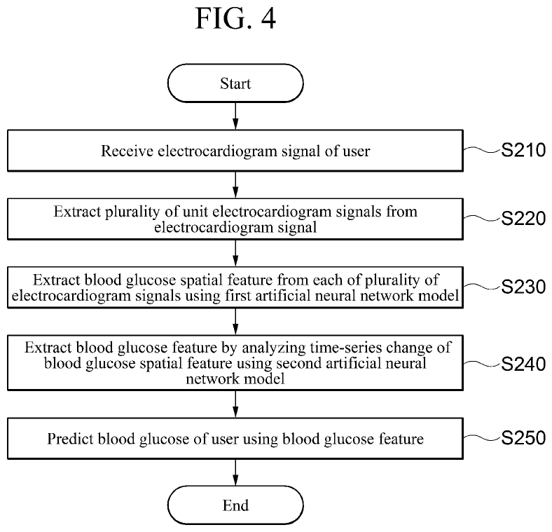

FIG. 4 is a flowchart illustrating an electrocardiogram-based blood glucose measuring method using artificial intelligence according to an exemplary embodiment of the present invention. Referring to FIG. 4, first, an electrocardiogram signal of a user measured by the electrocardiogram measuring device 10 may be received (S210).

Here, the electrocardiogram signal of the user measured by the electrocardiogram measuring device 10 may be as illustrated in FIG. 2, and may include a plurality of unit electrocardiogram signals according to a heartbeat of the user.

In addition, the plurality of unit electrocardiogram signals may be extracted from the electrocardiogram signal of the user (S220). Specifically, in S220, the plurality of unit electrocardiogram signals maybe extracted by excluding signals that do not satisfy a predetermined condition from the plurality of unit electrocardiogram signals. Here, the signals that do not satisfy the predetermined condition may include an interference signal, a noise signal, and the like generated because the electrocardiogram measurement is not properly performed.

Meanwhile, the plurality of extracted unit electrocardiogram signals may be aligned in time sequence and input to a first artificial neural network model.

Then, a blood glucose spatial feature may be extracted from each of the plurality of unit electrocardiogram signals using the first artificial neural network model (S220). Here, the first artificial neural network model may be a convolution neural network (CNN) model.

The convolution neural network model may extract a blood glucose spatial feature representing a spatial structure of an electrocardiogram signal for blood glucose from each of images of the plurality of unit electrocardiogram signals. That is, the blood glucose spatial feature may be a spatial feature related to a blood glucose level and/or a change in blood glucose in a waveform of the image of the unit electrocardiogram signal.

Then, a blood glucose feature may be extracted by analyzing a time series change of the blood glucose spatial feature using a second artificial neural network model (S240). Here, the second artificial neural network model may be a recurrent neural network (RNN) model.

The recurrent neural network model may extract a blood glucose feature of each of the images of the plurality of unit electrocardiogram signals based on a change in blood glucose spatial feature of each of the images of the plurality of unit electrocardiogram signals according to a time sequence of images of the plurality of unit electrocardiogram signals. Here, the blood glucose feature may be data used to predict a blood glucose level value and/or a blood glucose state value of the user.

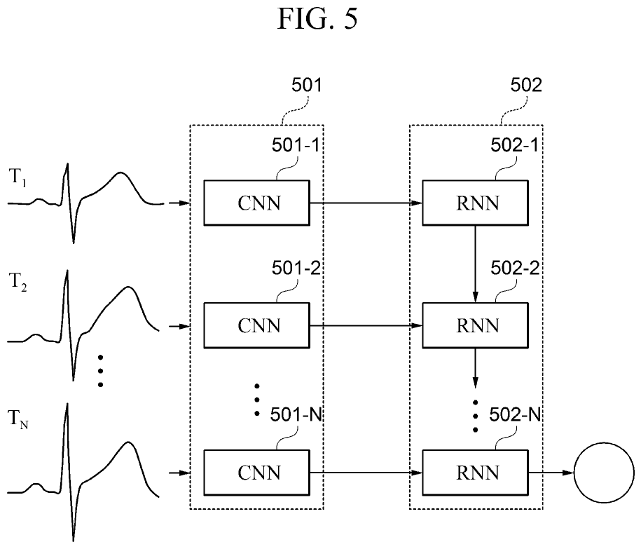

The artificial neural network model according to an exemplary embodiment of the present invention will be described in more detail with reference to FIG. 5.

FIG. 5 is a diagram illustrating an artificial neural network model according to an exemplary embodiment of the present invention.

Referring to FIG. 5, 200 unit electrocardiogram signals t₁, t₂, . . . t_{N} corresponding to 200 heartbeats may be extracted from the electrocardiogram signals at 5-minute intervals. Here, the number of unit electrocardiogram signals is only an example, and is not limited thereto.

In addition, the artificial neural network model may input a plurality of unit electrocardiogram signals (t₁, . . . t₂₀₀) extracted from the electrocardiogram signal to a convolution neural network model 501, and input an output of the convolution neural network model 501 to a recurrent neural network model 502.

Specifically, the convolution neural network model 501 may include the convolution neural networks corresponding to the number of the plurality of unit electrocardiogram signals (t₁, . . . t_{N}) extracted from the electrocardiogram signal, and images of the plurality of unit electrocardiogram signals may be input to each of the convolution neural networks 501-1, 501-2, . . . 501-N. Here, the image of the unit electrocardiogram signal may refer to the same image as the image of the waveform of the unit electrocardiogram.

That is, a first convolution neural network 501-1 may receive an image tₒ of a first unit electrocardiogram signal, a second convolution neural network 501-2 may receive an image t₁ of a second unit electrocardiogram signal, and an N-th convolution neural network 501-N may receive an image t_{N} of an N-th unit electrocardiogram signal.

In addition, each of the plurality of convolution neural networks 501-1, . . . 501-N may analyze the input image of the unit electrocardiogram signal and extract features of the image. That is, each of the plurality of convolution neural networks 501-1, . . . 501-N may include a plurality of layers, and each layer may receive the image of the unit electrocardiogram signal and process input data of the corresponding layer to generate output data.

Specifically, each of the plurality of convolution neural networks 501-1, . . . 501-N may include a convolutional layer that outputs a feature map as output data through a convolution operation on the input image. Among these convolutional layers, initial layers may operate to extract low-level features from the input, and upper layers may operate to extract more complex features, such as spatial features of the electrocardiogram signal associated with blood glucose.

In addition, the convolution neural networks 501-1, . . . 501-N may include pooling layers on which a pooling operation is performed in addition to the convolutional layers on which the convolution operation is performed. Here, the pooling layer may perform a pooling operation that reduces a spatial size of data in a corresponding region through max pooling that selects a maximum value in the region, average pooling that selects an average value of the region, or the like.

In addition, the convolution neural network 501-1, . . . 501-N may include a fully-connected layer that transforms each layer into a one-dimensional vector and connects the layers converted into one-dimensional vectors into one vector. Here, the fully-connected layer may determine classification.

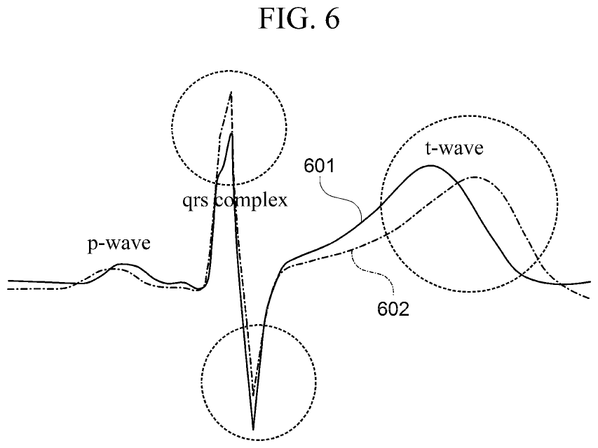

The convolution neural networks 501-1, . . . 501-N according to the present invention may extract blood glucose spatial features related to the blood glucose level and a change in blood glucose in the images of the unit ECG signals through the convolutional layer, the pooling layer, and the fully-connected layer. This will be described with reference to FIG. 6.

FIG. 6 is a diagram illustrating a relationship between an electrocardiogram signal and blood glucose according to an exemplary embodiment of the present invention. Referring to

FIG. 6, an electrocardiogram signal 601 is an electrocardiogram signal in a normal blood glucose, and an electrocardiogram signal 602 is an electrocardiogram signal in a low blood glucose.

It is determined that the concentration of blood glucose affects the electrical activity of the heart, and in particular, there is a correlation between the blood glucose and a change in amplitude of the QT interval the ST interval, and the QRS complex, and a significant change of the T wave.

The artificial neural network model according to the present invention may train a correlation between at least one of the blood glucose level value, the change in blood glucose, and the blood glucose state value and the electrocardiogram signal.

In particular, the convolution neural networks 502-1, . . . 502-N according to the present invention may extract the blood glucose spatial features corresponding to the change in amplitude of the QT interval, the ST interval, and the QRS complex, and the T wave.

On the other hand, the recurrent neural network model 502 may include recurrent neural networks corresponding to the number of the plurality of convolution neural networks 501-1, . . . 501-N, and each of the outputs of the plurality of convolution neural networks 501-1 . . . 501-N may be input to each of the recurrent neural networks 502-1 . . . 502-N. That is, a first recurrent neural network 502-1 may receive the output of the first convolution neural network 501-1, a second recurrent neural network 502-2 may receive the output of the second convolution neural network 501-2, and an N-th recurrent neural network 502-N may receive the output of the N-th convolution neural network 501-N.

Here, the recurrent neural network (RNN) is a deep learning technique that simultaneously considers current data and past data, and may simultaneously consider the current data and the past data by configuring not only the output of the convolution neural network 501 but also the connection between units constituting the artificial neural network as a directed cycle.

The recurrent neural network model 502 may extract the blood glucose feature according to a time series change of the blood glucose spatial feature by training the blood glucose spatial feature of each of the plurality of unit electrocardiogram images extracted through the plurality of convolution neural networks 501-1, . . . 501-N in time sequence.

Specifically, each of the plurality of recurrent neural networks 502-1, . . . 502-N may extract a blood glucose feature vector representing a time-series change of the blood glucose spatial feature of each of the plurality of unit electrocardiogram images extracted through the plurality of convolution neural networks 501-1, . . . 501-N. In addition, the recurrent neural network 502-N may extract the blood glucose feature calculated by synthesizing the extracted blood glucose features.

In addition, when the blood glucose feature is extracted from each of the images of the plurality of unit electrocardiogram signal, the recurrent neural network model 502 may predict a blood glucose based on the blood glucose feature (S250). Here, the predicted blood glucose may be a blood glucose level value and/or a blood glucose state value.

As an example, when the blood glucose is predicted as the blood glucose state value, the recurrent neural network model may classify the blood glucose features into a high blood glucose class, a medium blood glucose class, a low blood glucose class, and a normal blood glucose class, and may predict the blood glucose state value by determining to which class the blood glucose features derived from the images of the plurality of unit electrocardiogram signals belongs more.

In this case, the blood glucose level value and/or the blood glucose state value may be predicted by using soft voting in which a final prediction is determined by voting by combining a plurality of classifiers trained from the same data of the recurrent neural network model 502.

Here, a model used for soft voting may include at least two of random forests (RF), logistic regression, k-nearest neighbors (KNN), and LightGBM.

Meanwhile, according to the above-described example, the recurrent neural network model 502 has been described as using the RNN as an example, but according to another implement example of the present invention, the recurrent neural network model 502 may be implemented as a long short-term memory (LSTM).

According to the present invention, by presenting the artificial neural network model trained to predict the blood glucose of the user based on the extracted features, the blood glucose of the user may be more easily and quickly predicted.

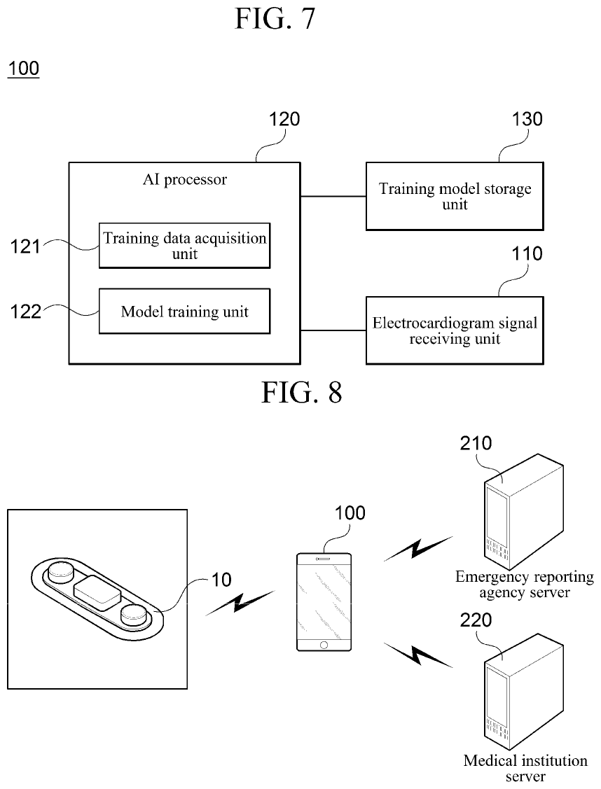

FIG. 7 is a block diagram illustrating an electrocardiogram-based blood glucose measuring apparatus using artificial intelligence according to an exemplary embodiment of the present invention. An electrocardiogram-based blood glucose measuring apparatus 100 according to the present invention may predict the blood glucose from the electrocardiogram signal of the user by training the artificial neural network model and using the trained artificial neural network model as described above.

The electrocardiogram-based blood glucose measuring apparatus 100 according to the present invention includes an electrocardiogram signal receiving unit 110, an AI processor 120, and a training model storage unit 130.

Here, the AI processor 120 may include a training data acquisition unit 121 and a model training unit 122.

The training data acquisition unit 121 may acquire training data required for a neural network model for classifying and recognizing data. Specifically, the training data acquisition unit 121 may acquire training data including electrocardiogram data and blood glucose data.

The model training unit 122 may train the artificial neural network model to have a criterion for determining how to classify predetermined data using the training data acquired through the training data acquisition unit 121. In this case, the model training unit 122 may train the neural network model using a training algorithm including supervised learning, unsupervised learning, reinforcement learning, error back-propagation, or gradient descent.

In addition, when the artificial neural network model is trained, the AI processor 120 may store the trained artificial neural network model in the training model storage unit 130.

Here, the artificial neural network model according to the present invention may be a combination of a convolution neural network (CNN) model and a recurrent neural network (RNN) model as illustrated in FIG. 5.

Meanwhile, the electrocardiogram-based blood glucose measuring apparatus 100 according to the present invention may predict the blood glucose from the electrocardiogram signal of the user by using the trained artificial neural network model. Specifically, the electrocardiogram signal receiving unit 110 may receive the electrocardiogram signal of the user measured by the electrocardiogram measuring device 10, and extract a plurality of unit electrocardiogram signals from the electrocardiogram signal of the user.

In this case, the AI processor 120 may predict blood glucose from the received electrocardiogram signal using the artificial neural network model stored in the training model storage unit 130.

Specifically, the plurality of extracted unit electrocardiogram signals may be aligned in time sequence and input to a convolution neural network model, and the convolution neural network model may extract a blood glucose spatial feature representing a spatial structure of an electrocardiogram signal for blood glucose from each of the images of the plurality of unit electrocardiogram signals.

In addition, the extracted blood glucose spatial feature may be input to a recurrent neural network model, and the recurrent neural network model may extract a blood glucose feature by analyzing a time series change of the blood glucose spatial feature. In addition, the recurrent neural network model may predict a blood glucose level value and/or a blood glucose state value based on the extracted blood glucose feature.

The electrocardiogram-based blood glucose measuring apparatus 100 according to the present invention as described above may be implemented using software, hardware, or a combination thereof. As an example, according to hardware implementation, the electrocardiogram-based blood glucose measuring apparatus 100 may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or electric units for performing other functions.

FIG. 8 is a block diagram illustrating a customized healthcare service system according to an exemplary embodiment of the present invention. Referring to FIG. 8, a customized healthcare service system 1000 may include an electrocardiogram measuring device 10, an electrocardiogram-based blood glucose measuring apparatus 100, an emergency reporting agency server 210, and a medical institution server 220.

The electrocardiogram measuring device 10 may be worn on or attached to the body of the user to measure the electrocardiogram of the user. Here, an implementation example of the electrocardiogram measuring device 10 may be a patch type as illustrated in FIG. 8. However, the electrocardiogram measuring device 10 is not limited thereto, and may have various shapes such as a watch type, a bracelet, a band, a ring, a necklace, an anklet, or a waistband.

The electrocardiogram-based blood glucose measuring apparatus 100 may receive the electrocardiogram signal measured by the electrocardiogram measuring device 10, input the received electrocardiogram signal to the trained artificial neural network model, and predict the blood glucose from the electrocardiogram signal. Since the detailed operation of training the artificial neural network model of the electrocardiogram-based blood glucose measuring apparatus 100 and predicting the blood glucose using the trained model has been described above, a detailed description thereof will be omitted.

Meanwhile, the electrocardiogram-based blood glucose measuring apparatus 100 may display the predicted blood glucose through a display unit or output the predicted blood glucose as a sound through audio.

In addition, when the blood glucose of the user is determined to be in a dangerous state such as high blood glucose or low blood glucose based on the predicted blood glucose of the user, the electrocardiogram-based blood glucose measuring apparatus 100 may transmit an emergency notification request including the blood glucose data of the user to the emergency reporting agency server 210 or may transmit an emergency prescription request including the blood glucose data of the user to the medical institution server 220.

In this case, the emergency reporting agency server 210 may determine the emergency degree of the user and the location of the user based on a blood glucose level value included in the blood glucose data of the user, and may provide an emergency dispatch service to the location of the user according to an emergency level of the user. Here, the emergency reporting agency server 210 may be a server such as 911.

In addition, the medical institution server 220 may provide a user-customized prescription service based on the blood glucose level value included in the blood glucose data of the user.

Here, an implementation example of the electrocardiogram-based blood glucose measuring apparatus 100 may be a smartphone as illustrated in FIG. 8. However, the electrocardiogram-based blood glucose measuring apparatus 100 is not limited thereto, and may be implemented in various forms, such as a tablet computer, a notebook computer, and the like.

Meanwhile, it has been described in FIG. 8 as an example that the electrocardiogram measuring device 10 and the electrocardiogram-based blood glucose measuring apparatus 100 are implemented as separate modules, but the present invention is not limited thereto, and the electrocardiogram measuring device 10 may also be implemented to perform all or some functions of the electrocardiogram-based blood glucose measuring apparatus 100.

According to the present invention described above, when the user is wearing the electrocardiogram measuring device, the healthcare system may provide the user-customized healthcare service such as providing the alarm to the user or automatically reporting to the emergency reporting agency in case of an emergency by predicting the blood glucose of the user based on the electrocardiogram of the user measured in real time.

The above description is merely illustrative of the technical idea of the present invention, and various modifications, changes and substitutions are possible within the scope that does not depart from the essential characteristics of the present invention by those of ordinary skill in the art to which the present invention pertains.

Accordingly, the exemplary embodiments disclosed in the present invention and the accompanying drawings are intended to explain, not to limit the technical spirit of the present invention, and the scope of the technical spirit of the present invention is not limited by these exemplary embodiments and the accompanying drawings. The protection scope of the present invention should be interpreted by the following claims, and all technical ideas within the equivalent scope should be interpreted as being included in the scope of the present invention.

Meanwhile, the method according to various exemplary embodiments of the present invention described above may be implemented as a program and provided to servers or devices. Accordingly, each apparatus may download the program by accessing the server or device in which the program is stored.

In addition, the method according to various exemplary embodiments of the present invention described above may be implemented as programs and be provided in a state in which it is stored in various non-transitory computer readable media. The non-transitory readable medium is not a medium that stores data for a short time such as a register, a cache, a memory, or the like, but means a machine readable medium that semi-permanently stores data. Specifically, various applications or programs described above may be stored and provided in the non-transitory computer readable medium such as a compact disk (CD), a digital versatile disk (DVD), a hard disk, a Blu-ray disk, a universal serial bus (USB), a memory card, a read only memory (ROM), or the like.

Although the exemplary embodiments of the present invention have been illustrated and described hereinabove, the present invention is not limited to the specific exemplary embodiments described above, but may be variously modified by those skilled in the art to which the present invention pertains without departing from the scope and spirit of the present invention as claimed in the claims. These modifications should also be understood to fall within the technical spirit and scope of the present invention.

1-10. (canceled)

11. A method for measuring electrocardiogram-based blood glucose using artificial intelligence, the method comprising:
receiving an electrocardiogram signal of a user;
extracting a plurality of unit electrocardiogram signals from the received electrocardiogram signal;
extracting a blood glucose spatial feature from each of the plurality of unit electrocardiogram signals using a first artificial neural network model; and
extracting a blood glucose feature by analyzing a time series change of the blood glucose spatial feature using a second artificial neural network model, and predicting blood glucose of the user based on the extracted blood glucose feature.

12. The method of claim 11, wherein the first artificial neural network model is a convolution neural network (CNN) model, and
the second artificial neural network model is a recurrent neural network (RNN) model.

13. The method of claim 12, wherein the convolution neural network model extracts the blood glucose spatial feature, which is a spatial feature related to a blood glucose level and/or a blood glucose change, from a waveform of each of the plurality of unit electrocardiogram signals.

14. The method of claim 13, wherein the number of convolution neural networks of the convolution neural network model coincides with the number of the plurality of extracted unit electrocardiogram signals.

15. The method of claim 14, wherein the recurrent neural network model extracts a blood glucose feature vector representing a time-series change of a blood glucose spatial feature of each of a plurality of unit electrocardiogram images extracted through the plurality of convolution neural networks, and synthesizes the extracted blood glucose features to extract the blood glucose feature.

16. The method of claim 15, wherein the number of recurrent neural networks of the recurrent neural network model coincides with the number of convolution neural networks.

17. The method of claim 12, wherein the predicted blood glucose is a blood glucose level value and/or a blood glucose state value.

18. An apparatus for measuring electrocardiogram-based blood glucose using artificial intelligence, the apparatus comprising:
an electrocardiogram signal receiving unit configured to receive an electrocardiogram signal of a user and extract a plurality of unit electrocardiogram signals from the received electrocardiogram signal; and
an AI processor configured to extract a blood glucose spatial feature from each of the plurality of unit electrocardiogram signals using a first artificial neural network model, extract a blood glucose feature by analyzing a time series change of the blood glucose spatial feature using a second artificial neural network model, and predict blood glucose of the user based on the extracted blood glucose feature.

19. The apparatus of claim 18, wherein the first artificial neural network model is a convolution neural network (CNN) model, and
the second artificial neural network model is a recurrent neural network (RNN) model.

20. The apparatus of claim 19, wherein the convolution neural network model extracts the blood glucose spatial feature, which is a spatial feature related to a blood glucose level and/or a blood glucose change, from a waveform of each of the plurality of unit electrocardiogram signals.

21. The apparatus of claim 20, wherein the number of convolution neural networks of the convolution neural network model coincides with the number of the plurality of extracted unit electrocardiogram signals.

22. The apparatus of claim 21, wherein the recurrent neural network model extracts a blood glucose feature vector representing a time-series change of a blood glucose spatial feature of each of a plurality of unit electrocardiogram images extracted through the plurality of convolution neural networks, and synthesizes the extracted blood glucose features to extract the blood glucose feature.

23. The apparatus of claim 22, wherein the number of recurrent neural networks of the recurrent neural network model coincides with the number of convolution neural networks.

24. The apparatus of claim 19, wherein the predicted blood glucose is a blood glucose level value and/or a blood glucose state value.

25. A user-customized healthcare service system comprising:
an electrocardiogram measuring device configured to measure an electrocardiogram of a user;
an electrocardiogram-based blood glucose apparatus configured to receive an electrocardiogram signal of the user from the electrocardiogram measuring device, extract a plurality of unit electrocardiogram signals from the received electrocardiogram signal, and predict blood glucose of the user from the plurality of unit electrocardiogram signals using a trained artificial neural network model; and
a server configured to provide an emergency dispatch service to a location of the user or provide a user-customized prescription service when the blood glucose of the user measured by the electrocardiogram-based blood glucose measuring apparatus is in a dangerous state,
wherein the electrocardiogram-based blood glucose measuring apparatus is configured to extract a blood glucose spatial feature from each of the plurality of unit electrocardiogram signals using a first artificial neural network model, extract a blood glucose feature by analyzing a time series change of the blood glucose spatial feature using a second artificial neural network model, and predict blood glucose of the user based on the extracted blood glucose feature.

26. The user-customized healthcare service system of claim 25, wherein the first artificial neural network model is a convolution neural network (CNN) model, and
the second artificial neural network model is a recurrent neural network (RNN) model.

27. The user-customized healthcare service system of claim 26, wherein the convolution neural network model extracts the blood glucose spatial feature, which is a spatial feature related to a blood glucose level and/or a blood glucose change, from a waveform of each of the plurality of unit electrocardiogram signals.

28. The user-customized healthcare service system of claim 27, wherein the number of convolution neural networks of the convolution neural network model coincides with the number of the plurality of extracted unit electrocardiogram signals.

29. The user-customized healthcare service system of claim 28, wherein the recurrent neural network model extracts a blood glucose feature vector representing a time-series change of a blood glucose spatial feature of each of a plurality of unit electrocardiogram images extracted through the plurality of convolution neural networks, and synthesizes the extracted blood glucose features to extract the blood glucose feature.

30. The user-customized healthcare service system of claim 27, wherein the predicted blood glucose is a blood glucose level value and/or a blood glucose state value.
